(19) United States
(12) Patent Application Publication
(10) Pub. No.: US 2026/0241101 A1
(43) Pub. Date: Aug. 20, 2026

(54) Ambulatory Infusion Pump with Limited-Mode Capabilities

(71) Applicant: Tandem Diabetes Care, Inc., San Diego, CA (US)

(72) Inventors: Nickolas Sherer, Escondido, CA (US); Thomas Ulrich, San Diego, CA (US); Ricardo Rueda, San Diego, CA (US); John Corbett, San Diego, CA (US)

(21) Appl. No.: 19/540,047

(22) Filed: Feb. 13, 2026

Related U.S. Application Data
(60) Provisional application No. 63/758,690, filed on Feb. 14, 2025.

(51) Int. Cl. A61M 5/172

(52) U.S. Cl. CPC A61M 5/1723; A61M 2205/3334; A61M 2205/3592; A61M 2230/201

(57) ABSTRACT

The present disclosure regards an ambulatory infusion pump with limited-mode capabilities. The pump includes a pump mechanism, a wireless antenna, and a processor. The processor is configured to receive BGL data points via the wireless antenna. The processor is also configured to continuously determine whether a number of the BGL data points received within a previous amount of time satisfies a criterion for closed-loop operation or a criterion for limited operation. Additionally, the processor is configured to administer the medicament in a closed-loop mode if the closed-loop criterion is satisfied and administer the medicament in a limited mode if the limited-mode criterion is satisfied. Further, the processor is configured to administer the medicament in an open-loop mode if neither of the criteria is satisfied.

### RELATED APPLICATIONS

This application claims priority to U.S. Provisional Patent App. No. 63/758,690 (filed Feb. 14, 2025). This application also relates to U.S. Provisional Patent App. Nos. 63/429,407 (filed Dec. 1, 2022), 63/615,481 (filed Dec. 28, 2023), and 63/615,579 (filed Dec. 28, 2023) and W.O. Patent App. No. PCT/US2023/082084 (filed Dec. 1, 2023). Each of the foregoing applications is hereby incorporated in its entirety.

### TECHNICAL FIELD

The present disclosure relates, generally, to medical devices and, more specifically, to ambulatory infusion pumps for diabetes management.

### BACKGROUND

Ambulatory infusion pumps require a relatively steady stream of data in order to operate competently in a closed-loop manner (i.e., absent user intervention). Accordingly, without a critical mass of data, many infusion pumps will revert to open-loop delivery in order to avoid over-and under-infusion. This approach to medicament delivery means that such infusion pumps occasionally operate in open-loop mode despite having received useful data.

### SUMMARY

The present disclosure seeks to refine ambulatory infusion pump operation by introducing an intermediary “limited mode” for bridging the gap between closed-and-open loop operation. In instances where a pump has received data that is insufficient for operating in a closed-loop mode, the pump can instead operate in limited mode—rather than ignoring the data altogether and reverting to open-loop operation.

As with closed-loop operation, a pump operating in limited mode will continue to determine how best to administer a medicament (e.g., insulin, glucagon). However, while in limited mode, the pump will also operate within one or more guardrails prescribed thereby, such as a maximum flow rate. The present disclosure provides various examples of said guardrails and also elaborates on the boundaries between closed-loop, limited, and open-loop modes.

Exemplary embodiments include the following:

An Ambulatory Infusion Pump. The ambulatory infusion pump includes a pump mechanism, a wireless antenna, and a processor. The pump mechanism is configured to deliver a medicament to a user. The wireless antenna is configured to receive blood-glucose level (BGL) data points regarding a BGL of the user. The processor is configured to receive the BGL data points via the wireless antenna. The processor is further configured to continuously determine whether a number of the BGL data points received within a previous amount of time satisfies a criterion for closed-loop operation or a criterion for limited operation. Additionally, the processor is configured to, responsive to determining the number of BGL data points satisfies the criterion for closed-loop operation, administer the medicament in a closed-loop mode. Further, the processor is configured to, responsive to determining the number of BGL data points satisfies the criterion for limited operation but not the criterion for closed-loop operation, administer the medicament in a limited mode. Moreover, the processor is configured to, responsive to determining the number of BGL data points satisfies neither the criterion for closed-loop operation nor the criterion for limited operation, administer the medicament in an open-loop mode.

A Non-Transitory, Computer-Readable Medium. The computer-readable medium stores instructions that, when executed by a processor of an electronic device, cause the electronic device to perform operations. The operations include receiving, via a wireless antenna, BGL data points regarding a BGL of a user. The operations also include continuously determining whether a number of the BGL data points received within a previous amount of time satisfies a criterion for closed-loop operation or a criterion for limited operation. Additionally, the operations include, responsive to determining the number of BGL data points satisfies the criterion for closed-loop operation, administering a medicament in a closed-loop mode. Further, the operations include, responsive to determining the number of BGL data points satisfies the criterion for limited operation but not the criterion for closed-loop operation, administering the medicament in a limited mode. Moreover, the operations include, responsive to determining the number of BGL data points satisfies neither the criterion for closed-loop operation nor the criterion for limited operation, administering the medicament in an open-loop mode.

A Computer-Implemented Method. The computer-implemented method is a method for operating an ambulatory infusion pump in a limited mode. The method includes receiving, via a wireless antenna, BGL data points regarding a BGL of a user. The method also includes continuously determining whether a number of the BGL data points received within a previous amount of time satisfies a criterion for closed-loop operation or a criterion for limited operation. Additionally, the method includes, responsive to determining the number of BGL data points satisfies the criterion for closed-loop operation, administering a medicament in a closed-loop mode. Further, the method includes, responsive to determining the number of BGL data points satisfies the criterion for limited operation but not the criterion for closed-loop operation, administering the medicament in a limited mode. Moreover, the method includes, responsive to determining the number of BGL data points satisfies neither the criterion for closed-loop operation nor the criterion for limited operation, administering the medicament in an open-loop mode.

Based on the following Detailed Description, other configurations of the subject technology will be apparent to those skilled in the art. The Detailed Description describes various configurations of the subject technology, particularly with respect to illustrations thereof. Notwithstanding, the subject technology is capable of other and different configurations, and its several details are capable of modification in various other respects—all without departing from the scope of the subject technology. The Drawings and Detailed Description are therefore presented as illustrative in nature and should not be construed as restricting the present disclosure.

### BRIEF DESCRIPTION OF THE DRAWINGS

For a better understanding of the present disclosure, reference should be made to the Detailed Description, below, in conjunction with the following drawings. Like reference numerals refer to corresponding parts throughout the figures and the description.

FIGS. 1A and 1B are system diagrams that illustrate example ambulatory infusion systems for medicament delivery, according to various aspects of the subject technology.

FIG. 2 documents example events occurring at an ambulatory infusion pump, including the receipt of BGL data and shifts by the pump between open-loop, limited, and closed-loop modes, according to various aspects of the subject technology.

FIG. 3 illustrates an example of limited mode operation by an ambulatory infusion pump, according to various aspects of the subject technology.

FIG. 4 illustrates an example process for operating an ambulatory infusion pump in a limited mode, according to various aspects of the subject technology.

### DETAILED DESCRIPTION

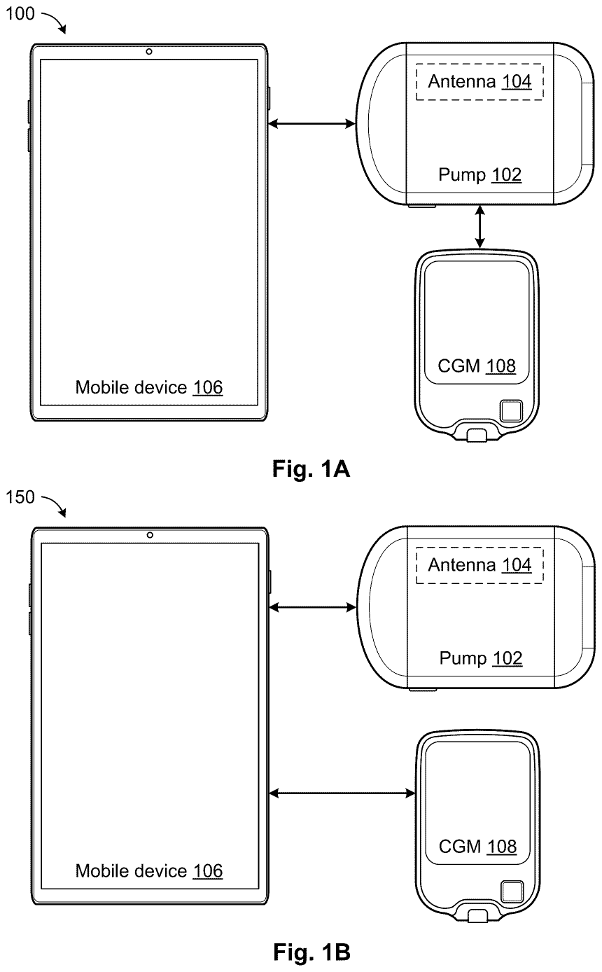

FIGS. 1A and 1B illustrate example ambulatory infusion systems 100 and 150 for medicament delivery, according to various aspects of the subject technology. Each system 100 and 150 includes an ambulatory infusion pump 102, a mobile device 106, and a continuous glucose monitor (CGM) 108.

In both systems 100 and 150, the pump 102 is configured to deliver a medicament to a user based at least in part on data or instructions received via its wireless antenna 104. For example, the pump 102 can receive BGL data and then determine a flow rate for medicament delivery based on the data. Or, as another example, the pump 102 can receive a bolus request and administer a bolus in accordance therewith. In some embodiments, the pump 102 is configured to store and deliver insulin to the user for BGL management; though, it can also be configured to deliver glucagon or another medicament for BGL management.

The mobile device 106 is configured to communicate with the pump 102. This communication may include the mobile device 106 sending instructions (e.g., a bolus request) or physiological parameters (or adjustments thereto) to the pump 102. The communication can also include the pump 102 sending notifications or status information back to the mobile device 106, which the mobile device 106 can then display for the user. In some embodiments, the mobile device 106 is a smartphone, a tablet, a smartwatch, or a laptop computer.

The CGM 108 is configured to collect data regarding the user's BGL and transmit the data to the pump 102, the mobile device 106, or a server in communication with at least one of the devices 102 or 106. In some embodiments, the CGM 108 collects the BGL data at regular intervals (e.g., every five minutes). Additionally, in some embodiments, the CGM 108 also transmits additional information regarding the BGL data to one of the aforesaid devices, such as a metric indicative of noise in the data.

In the first system 100, the pump 102 is in communication with both the mobile device 106 and the CGM 108. In this system 100, the pump 102 is configured to execute an algorithm for closed-loop operation based on BGL data from the CGM 108 and physiological parameters from the mobile device 106. By contrast, in the second system 150, the pump 102 is in communication only with the mobile device 106, which itself communicates with the CGM 108. In this system, either the pump 102 or the mobile device 106 may run the algorithm for medicament delivery. If the mobile device 106 manages the algorithm, it will send instructions (e.g., flow rate, bolus parameters) to the pump 102 for execution.

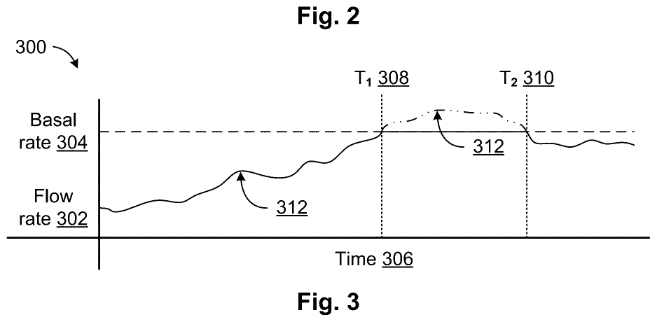

FIG. 2 describes example events occurring at an ambulatory infusion pump (e.g., pump 102), including the receipt of BGL data and shifts by the pump between open-loop, limited, and closed-loop modes, according to various aspects of the subject technology. As noted above, there exists a gap between closed-and open-loop operation, with the former requiring a critical mass of BGL data and the latter requiring no BGL data whatsoever. The limited mode described herein bridges this gap—allowing ambulatory infusion pumps to use BGL data productively, even when said data is insufficient for competent closed-loop operation.

As an example, consider two points of BGL data: first, a data point errantly indicating that the BGL level of a user is significantly higher than it actually is (e.g., 200 mg/dL versus 120 mg/dL); and second, a data point correctly indicating that the BGL level of the user is particularly low (e.g., 58 mg/dL).

A pump operating in closed-loop mode and having only the first data point would likely administer too much of a BGL-reducing drug (e.g., insulin) based on the inaccurate data. This can be avoided by requiring a certain amount of BGL data for closed-loop operation because BGL-data deviations tend to dissipate across multiple data points. However, such a requirement would lead to a second issue, where the pump has only the second point of data. In this scenario, the pump would operate in open-loop mode due to the lack of multiple data points and, in so doing, would likely administer too much of the BGL-reducing drug by failing to account for the low BGL of the user indicated by the single data point.

Limited mode addresses these two scenarios by allowing the ambulatory infusion pump to continue making data-driven determinations while limiting said determinations to account for the reduced amount of available data. A pump operating in an exemplary limited mode, for instance, will determine medicament flow rates based on available data but will ensure the medicament is never administered at a rate higher than a programmed basal rate; in the event the determined flow rate exceeds the programmed basal rate, the pump will instead deliver the medicament at the programmed basal rate.

In this manner, the pump can properly handle both of the aforenoted BGL data points. If it has only the first data point, the pump will avoid over-infusion by limiting medicament delivery to the programmed basal rate. And, if it has only the second data point, the pump will likewise avoid over-infusion by slowing or stopping medicament delivery accordingly. In some embodiments, the pump will operate in limited mode if it has received only one or two BGL data points in the past thirty minutes. Variations on the limited mode are possible, as discussed in more detail herein.

Returning to FIG. 2, the event table 200 includes four columns: The first column 202 indicates times at which the aforenoted events occur at the ambulatory infusion pump. The second column 204 includes descriptions of the events. The third column 206 indicates how many BGL data points the pump has received within the past thirty minutes. And the fourth column 208 indicates the current operating mode of the pump.

The ambulatory infusion pump at which the events occur is configured to receive BGL data from a CGM (e.g., CGM 108) or from another device (e.g., mobile device 106, a remote server) connected thereto. In order to operate in closed-loop mode, the pump requires a certain amount of recent BGL data. This is to ensure the pump has enough data for determining how to administer a medicament to the user.

In the illustrated embodiment, the pump will not operate in closed-loop mode unless it has received at least three BGL data points within the last thirty minutes. On the other hand, if the pump has not received any BGL data within the last thirty minutes, then the pump will operate in open-loop mode. And if the pump has received some BGL data but less than the required amount for closed-loop operation, then the pump will operate in limited mode rather than reverting to open-loop operation. This allows the pump to use received BGL data productively while working within safety boundaries prescribed by the limited mode (see FIG. 3).

In some embodiments, the pump uses other metrics or timeframes for determining whether to operate in closed-loop or limited modes. For instance, the pump may require two, four, or more BGL data points within a certain amount of time (e.g., fifteen, twenty, or sixty minutes) before operating in closed-loop mode. As another example, the pump may require at least two, or even three BGL data points within a certain amount of time (e.g., fifteen, twenty, or sixty minutes) before it will operate in a limited mode.

These metrics and timeframes can be pre-programmed; however, in some embodiments, they are adjusted based on one or more parameters. For instance, if the BGL data is particularly noisy, the pump may require more data or decrease the timeframe based on a noise metric indicative of the noise of the data points. As another example, the BGL data can also be adjusted based on physiological parameters of the patient (e.g., basal rate, correction factor, carb ratio) or infusion parameters of the pump (e.g., flow rate, bolus size).

As documented in the event table 200, the pump does not receive any BGL data from 8:00 a through 9:00 a. Quiet periods like this are common for CGM devices when they first warm up, which usually takes an hour or two. During this time without any BGL data, the pump operates in open-loop mode.

At 10:07 a, however, the pump receives BGL data and switches from open-loop mode into limited mode. It continues to receive BGL data and operate in limited mode until 10:17 a, at which point it has received three BGL data points in the last thirty minutes (at 10:07 a, 10:12 a, and 10:17 a). Accordingly, the pump switches to closed-loop operation at 10:17 a.

The pump operates in closed-loop mode through minor data shortages between 11:07 a and 11:22 a and again between 11:22 a and 11:37 a because, notwithstanding those shortages, the pump has received at least three recent BGL data points throughout this period of time. Data shortages at the pump can be caused by connectivity issues between the pump and the CGM, software or firmware glitches, device synchronization issues, interference from other electronic devices, a malfunctioning CGM sensor, or obstruction between the pump and the CGM.

As of 11:38 a, the pump has only received two BGL data points in the last thirty minutes (at 11:22 a and 11:37 a), so it switches back into limited mode. The pump does not receive additional BGL data until 12:02 p, at which point BGL data is once again received every five minutes. Accordingly, the pump resumed closed-loop operation at 12:07 p after having received three BGL data points in the previous thirty minutes (at 11:38 a, 12:02 p, and 12:07 p). The pump continues to receive BGL data and maintains closed-loop operation accordingly.

FIG. 3 illustrates an example of limited mode operation by an ambulatory infusion pump (e.g., pump 102), according to various aspects of the subject technology. The graph 300 includes curves for the flow rate 302 of the pump, a programmed basal rate 304, and axes for time 306 and magnitude (e.g., in units per hour).

While operating in limited mode, the pump works within certain limitations to account for the reduced amount of BGL data it has received. As with closed-loop mode, the pump continues to determine a flow rate 312 for medicament delivery based on BGL data received from a CGM (e.g., CGM 108) or from a device connected thereto (e.g., mobile device 106, a remote server). However, the pump in the illustrated embodiment also ensures the medicament is not delivered at a rate higher than the programmed basal rate 304. This is an example guardrail for limited mode operation; other guardrails (e.g., a minimum flow rate) are within the scope of the present disclosure.

Prior to a first time T₁ 308, the determined flow rate 312 does not exceed the basal rate 304. Accordingly, the pump administers the medicament at the determined flow rate 312 up until T₁ 308 (i.e., the actual flow rate 302 matches the determined flow rate 312). At T₁ 308, however, the determined flow rate 312 exceeds the programmed basal rate 304. Rather than administer the medicament at the determined flow rate 312, the pump limits its flow rate 302 to the programmed basal rate 304 in order to avoid over-infusion. This continues until a second time T₂ 310, at which point the determined flow rate 312 no longer exceeds the basal rate 304. Accordingly, at T2 310, the pump resumes medicament delivery at the determined flow rate 312.

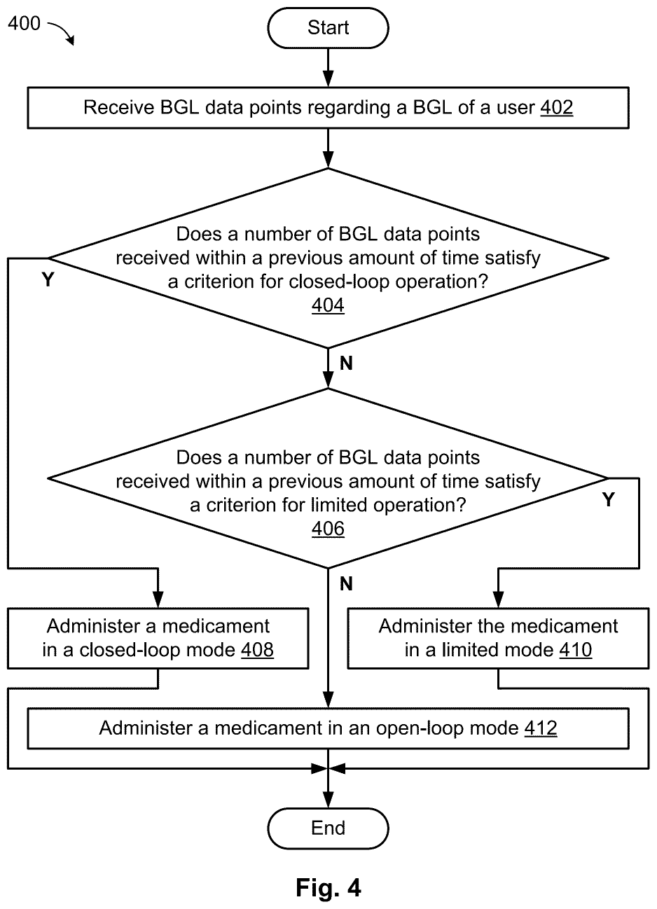

FIG. 4 illustrates an example process 400 for operating an ambulatory infusion pump in a limited mode, according to various aspects of the subject technology. The operations of the process 400 can be executed by devices discussed above with respect to FIGS. 1A-3, such as the ambulatory infusion pump 102 or mobile device 106. At least some of the operations can also be executed by a server connected to these devices 102 and 106.

In some embodiments, instructions stored in a non-transitory, computer-readable medium correspond to operations of the example process 400. In such embodiments, a processor of an electronic device (e.g., pump 102, mobile device 106) can execute the instructions to cause the electronic device to perform the corresponding operations.

The process 400 includes receiving (402) BGL data points regarding a BGL of a user, such as those discussed above for FIG. 2. For example, the BGL data points can be received via a wireless antenna (e.g., antenna 104) of an ambulatory infusion pump (e.g., pump 102). The BGL data points can be received, for instance, from a CGM (e.g., CGM 108) or from another device (e.g., mobile device 106, a remote server) connected thereto.

The process 400 also includes determining (404 and 406) whether a number of the BGL data points received within a previous amount of time (see, e.g., third column 206) satisfies a criterion for closed-loop operation or a criterion for limited operation. As an example, the former determination (404) may involve determining whether three or more BGL data points were received in the thirty minutes immediately preceding the determination. And the latter determination (406) can involve determining whether one or two BGL data points were received in the preceding thirty minutes.

Additionally, the process 400 includes, responsive to determining (404) the number of BGL data points satisfies the criterion for closed-loop operation, administering (408) a medicament in a closed-loop mode. For example, administering the medicament in the closed-loop mode can include determining a flow rate based on the BGL data points and causing the pump mechanism to deliver the medicament to the user at the flow rate.

Further, the process 400 includes, responsive to determining (406 and 404) the number of BGL data points satisfies the criterion for limited operation but not the criterion for closed-loop operation, administering (410) the medicament in a limited mode. For example, administering the medicament in the limited mode can include operating as in closed-loop mode while also ensuring the flow rate never exceeds a programmed basal rate. In some embodiments, limited mode uses a different metric for limiting maximum medicament output, such as a multiple of the programmed basal rate (e.g., 0.8 or 1.3 times) or a weighted average of the programmed basal rate and a determined flow rate.

Accordingly, in some embodiments, the process also includes receiving a basal rate (e.g., basal rate 304). In such embodiments, administering the medicament in the limited mode includes determining a flow rate (e.g., flow rate 312) for delivering the medicament to the user based on the BGL data points, determining whether the flow rate exceeds the basal rate, responsive to determining the flow rate does not exceed the basal rate, causing the pump mechanism to deliver the medicament to the user at the flow rate, and responsive to determining the flow rate exceeds the basal rate, causing the pump mechanism to deliver the medicament to the user at the basal rate. An example of this is illustrated in FIG. 3 and discussed in more detail above.

Moreover, the process 400 includes, responsive to determining (404 and 406) the number of BGL data points satisfies neither the criterion for closed-loop operation nor the criterion for limited operation, administering (412) the medicament in an open-loop mode. For example, administering the medicament in the open-loop mode can include forgoing determining the flow rate based on the BGL data points and causing the pump mechanism to deliver the medicament to the user at the basal rate.

In some embodiments, the process 400 involves continuously determining (404 and 406) whether the number of BGL data points received within the previous amount of time satisfies the criteria for closed-loop operation or limited operation. For instance, these determinations (404 and 406) can occur while also administering (408, 410, and 412) the medicament in the closed-loop, limited, or open-loop modes, the process. In this manner, the process 400 can account for changes in the number of BGL data points received over time (see, e.g., fourth column 208).

Additionally, in some embodiments, the process 400 includes establishing a connection with an electronic device (e.g., mobile device 106, CGM 108) via a wireless antenna (e.g., antenna 104), where receiving (402) the data points occurs via the wireless antenna and includes receiving the BGL data points from the electronic device. In such embodiments, the process 400 may further include detecting a failure in the connection with the electronic device and, responsive to detecting the failure, administering the medicament in the open-loop mode until the connection with the electronic device is reestablished.

Further, in some embodiments, the process further includes determining an amount of noise in the BGL data points and adjusting the amount of time, the criterion for closed-loop operation, or the criterion for limited operation based on the amount of BGL noise.

In some of these embodiments, the BGL data points are collected by a CGM (e.g., CGM 108), and the process 400 further includes receiving an identifier of the CGM, determining a noise rating for the CGM based on the identifier of the CGM. Accordingly, determining the amount of noise in the BGL data points can be based on the noise rating.

Also, in some of these embodiments, the BGL data points are collected by a CGM (e.g., CGM 108), and the process 400 further includes receiving an identifier of the CGM and determining a sampling rate for the CGM based on the identifier of the CGM. Accordingly, adjusting the amount of time, the criterion for closed-loop operation, or the criterion for limited operation can be further based on the sampling rate.

Moreover, in some embodiments, the number of BGL data points satisfying the criterion for limited operation but not the criterion for closed-loop operation is due to a CGM (e.g., CGM 108) transitioning from a warmup phase into a default phase, and the BGL data points are collected by the CGM.

Furthermore, in some embodiments, the number of BGL data points satisfying the criterion for limited operation but not the criterion for closed-loop operation is due to a failure in communication involving a CGM (e.g., CGM 108), and the BGL data points are collected by the CGM.

Illustrative Clauses. For further reference, example aspects of the present disclosure are included below as numbered clauses. These clauses are provided for illustrative purposes and are not intended to limit the subject technology.

Clause 1. An ambulatory infusion pump comprising: a pump mechanism configured to deliver a medicament to a user; a wireless antenna configured to receive BGL data points regarding a BGL of the user; and a processor configured to: receive the BGL data points via the wireless antenna; continuously determine whether a number of the BGL data points received within a previous amount of time satisfies a criterion for closed-loop operation or a criterion for limited operation; responsive to determining the number of BGL data points satisfies the criterion for closed-loop operation, administer the medicament in a closed-loop mode; responsive to determining the number of BGL data points satisfies the criterion for limited operation but not the criterion for closed-loop operation, administer the medicament in a limited mode; and responsive to determining the number of BGL data points satisfies neither the criterion for closed-loop operation nor the criterion for limited operation, administer the medicament in an open-loop mode.

Clause 2. The ambulatory infusion pump of clause 1, wherein: the processor is further configured to receive a basal rate; and administering the medicament in the limited mode comprises (i) determining a flow rate for delivering the medicament to the user based on the BGL data points, (ii) determining whether the flow rate exceeds the basal rate, (iii) responsive to determining the flow rate does not exceed the basal rate, causing the pump mechanism to deliver the medicament to the user at the flow rate, and (iv) responsive to determining the flow rate exceeds the basal rate, causing the pump mechanism to deliver the medicament to the user at the basal rate.

Clause 3. The ambulatory infusion pump of Clause 2, wherein: administering the medicament in the closed-loop mode comprises (i) determining the flow rate based on the BGL data points and (ii) causing the pump mechanism to deliver the medicament to the user at the flow rate; and administering the medicament in the open-loop mode comprises (i) forgoing determining the flow rate based on the BGL data points and (ii) causing the pump mechanism to deliver the medicament to the user at the basal rate.

Clause 4. The ambulatory infusion pump of clause 1, wherein the processor is further configured to: establish a connection with an electronic device via the wireless antenna, wherein receiving the BGL data points via the wireless antenna comprises receiving the BGL data points from the electronic device; detect a failure in the connection with the electronic device; and responsive to detecting the failure in the connection, administer the medicament in the open-loop mode until the connection with the electronic device is reestablished.

Clause 5. The ambulatory infusion pump of any one of clauses 1-4, wherein the processor is further configured to: determine an amount of noise in the BGL data points; and adjust the amount of time, the criterion for closed-loop operation, or the criterion for limited operation based on the amount of BGL noise.

Clause 6. The ambulatory infusion pump of clause 5, wherein: the BGL data points are collected by a CGM; the processor is further configured to (i) receive an identifier of the CGM and (ii) determine a noise rating for the CGM based on the identifier of the CGM; and determining the amount of noise in the BGL data points is based on the noise rating.

Clause 7. The ambulatory infusion pump of either clause 5 or 6, wherein: the BGL data points are collected by a CGM; the processor is further configured to (i) receive an identifier of the CGM and (ii) determine a sampling rate for the CGM based on the identifier of the CGM; and adjusting the amount of time, the criterion for closed-loop operation, or the criterion for limited operation is further based on the sampling rate.

Clause 8. The ambulatory infusion pump of any one of clauses 1-7, wherein: the previous amount of time is a previous thirty minutes; the number of the BGL data points received within the previous amount of time satisfies the criterion for closed-loop operation if the processor received three or more BGL data points in the previous thirty minutes; and the number of the BGL data points received within the previous amount of time satisfies the criterion for limited operation if the processor received one or two BGL data points in the previous thirty minutes.

Clause 9. The ambulatory infusion pump of any one of clauses 1-8, wherein the number of BGL data points satisfying the criterion for limited operation but not the criterion for closed-loop operation is due to a CGM transitioning from a warmup phase into a default phase, and the BGL data points are collected by the CGM.

Clause 10. The ambulatory infusion pump of any one of clauses 1-9, wherein the number of BGL data points satisfying the criterion for limited operation but not the criterion for closed-loop operation is due to a failure in communication involving a CGM, and the BGL data points are collected by the CGM.

Clause 11. A non-transitory, computer-readable medium storing instructions that, when executed by a processor of an electronic device, cause the electronic device to perform operations comprising: receiving, via a wireless antenna, BGL data points regarding a BGL of a user; continuously determining whether a number of the BGL data points received within a previous amount of time satisfies a criterion for closed-loop operation or a criterion for limited operation; responsive to determining the number of BGL data points satisfies the criterion for closed-loop operation, administering a medicament in a closed-loop mode; responsive to determining the number of BGL data points satisfies the criterion for limited operation but not the criterion for closed-loop operation, administering the medicament in a limited mode; and responsive to determining the number of BGL data points satisfies neither the criterion for closed-loop operation nor the criterion for limited operation, administering the medicament in an open-loop mode.

Clause 12. The non-transitory, computer-readable medium of clause 11, wherein: the operations further comprise receiving a basal rate; and administering the medicament in the limited mode comprises (i) determining a flow rate for delivering the medicament to the user based on the BGL data points, (ii) determining whether the flow rate exceeds the basal rate, (iii) responsive to determining the flow rate does not exceed the basal rate, causing a pump mechanism to deliver the medicament to the user at the flow rate, and (iv) responsive to determining the flow rate exceeds the basal rate, causing the pump mechanism to deliver the medicament to the user at the basal rate.

Clause 13. The non-transitory, computer-readable medium of clause 12, wherein: administering the medicament in the closed-loop mode comprises (i) determining the flow rate based on the BGL data points and (ii) causing the pump mechanism to deliver the medicament to the user at the flow rate; and administering the medicament in the open-loop mode comprises (i) forgoing determining the flow rate based on the BGL data points and (ii) causing the pump mechanism to deliver the medicament to the user at the basal rate.

Clause 14. The non-transitory, computer-readable medium of any one of clauses 11-13, wherein the operations further comprise: establishing a connection with an electronic device via the wireless antenna, wherein receiving the BGL data points via the wireless antenna comprises receiving the BGL data points from the electronic device; detecting a failure in the connection with the electronic device; and responsive to detecting the failure in the connection, administering the medicament in the open-loop mode until the connection with the electronic device is reestablished.

Clause 15. The non-transitory, computer-readable medium of any one of clauses 11-14, wherein the operations further comprise: determining an amount of noise in the BGL data points; and adjusting the amount of time, the criterion for closed-loop operation, or the criterion for limited operation based on the amount of BGL noise.

Clause 16. The non-transitory, computer-readable medium of clause 15, wherein: the BGL data points are collected by a CGM; the operations further comprise (i) receiving an identifier of the CGM and (ii) determining a noise rating for the CGM based on the identifier of the CGM; and determining the amount of noise in the BGL data points is based on the noise rating.

Clause 17. The non-transitory, computer-readable medium of either clause 15 or 16, wherein: the BGL data points are collected by a CGM; the operations further comprise (i) receiving an identifier of the CGM and (ii) determining a sampling rate for the CGM based on the identifier of the CGM; and adjusting the amount of time, the criterion for closed-loop operation, or the criterion for limited operation is further based on the sampling rate.

Clause 18. The non-transitory, computer-readable medium of any one of clauses 11-17, wherein: the previous amount of time is a previous thirty minutes; the number of the BGL data points received within the previous amount of time satisfies the criterion for closed-loop operation if three or more BGL data points were received in the previous thirty minutes; and the number of the BGL data points received within the previous amount of time satisfies the criterion for limited operation if one or two BGL data points were received in the previous thirty minutes.

Clause 19. The non-transitory, computer-readable medium of any one of clauses 11-18, wherein the number of BGL data points satisfying the criterion for limited operation but not the criterion for closed-loop operation is due to a CGM transitioning from a warmup phase into a default phase, and the BGL data points are collected by the CGM.

Clause 20. A computer-implemented method for operating an ambulatory infusion pump in a limited mode, the method comprising: receiving, via a wireless antenna, BGL data points regarding a BGL of a user; continuously determining whether a number of the BGL data points received within a previous amount of time satisfies a criterion for closed-loop operation or a criterion for limited operation; responsive to determining the number of BGL data points satisfies the criterion for closed-loop operation, administering a medicament in a closed-loop mode; responsive to determining the number of BGL data points satisfies the criterion for limited operation but not the criterion for closed-loop operation, administering the medicament in a limited mode; and responsive to determining the number of BGL data points satisfies neither the criterion for closed-loop operation nor the criterion for limited operation, administering the medicament in an open-loop mode.

Further Consideration. The specific order or hierarchy of steps in the processes disclosed herein is an illustration of example approaches. Based upon design preferences, it is understood that the specific order or hierarchy of steps in the processes may be rearranged. Some of the steps may be performed simultaneously. The accompanying method claims recite certain elements of the various steps in an exemplary order, and are not meant to be limited to the specific order or hierarchy presented.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Headings and subheadings, if any, are used for convenience only and do not limit the invention described herein.

The predicate words “configured to,” “operable to,” and “programmed to” do not imply any particular tangible or intangible modification of a subject, but rather are intended to be used interchangeably. For example, a processor configured to monitor and control an operation or a component may also mean the processor being programmed to monitor and control the operation or the processor being operable to monitor and control the operation. Likewise, a processor configured to execute code can be construed as a processor programmed to execute code or operable to execute code.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “implementation” does not imply that such implementation is essential to the subject technology or that such implementation applies to all configurations of the subject technology. A disclosure relating to an implementation may apply to all implementations, or one or more implementations. An implementation may provide one or more examples. A phrase such as “implementations” may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

As used herein, the terms “determine” and “determining” encompass a wide variety of actions. For example, “determining” may include calculating, computing, processing, deriving, generating, obtaining, looking up (e.g., looking up in a table, a database or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the term “message” encompasses a wide variety of formats for communicating (e.g., transmitting or receiving) information. A message may include a machine readable aggregation of information such as an XML document, fixed field message, comma separated message, JSON, a custom protocol, or the like. A message may, in some embodiments, include a signal utilized to transmit one or more representations of the information. While recited in the singular, it will be appreciated that a message may be composed, transmitted, stored, received, and so on in multiple parts.

As used herein, the term “selectively” or “selective” may encompass a wide variety of actions. For example, a “selective” process may include determining one option from multiple options. A “selective” process may include one or more of: dynamically determined inputs, preconfigured inputs, or user-initiated inputs for making the determination. In some embodiments, an n-input switch may be included to provide selective functionality where n is the number of inputs used to make the selection.

As used herein, the terms “correspond” or “corresponding” encompasses a structural, functional, quantitative, or qualitative correlation or relationship between two or more objects, data sets, information and the like, preferably where the correspondence or relationship may be used to translate one or more of the two or more objects, data sets, information and the like so to appear to be the same or equal. Correspondence may be assessed using one or more of a threshold, a value range, fuzzy logic, pattern matching, a machine-learning assessment model, or combinations thereof.

In any embodiment, data generated or detected can be forwarded to a “remote” device or location, where “remote,” means a location or device other than the location or device at which the program is executed. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but separated, or at least in different rooms or different buildings, and can be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet or including email transmissions and information recorded on websites and the like.

What is claimed is:

1. An ambulatory infusion pump comprising:
a pump mechanism configured to deliver a medicament to a user;
a wireless antenna configured to receive BGL data points regarding a BGL of the user; and
a processor configured to:
receive the BGL data points via the wireless antenna;
continuously determine whether a number of the BGL data points received within a previous amount of time satisfies a criterion for closed-loop operation or a criterion for limited operation;
responsive to determining the number of BGL data points satisfies the criterion for closed-loop operation, administer the medicament in a closed-loop mode;
responsive to determining the number of BGL data points satisfies the criterion for limited operation but not the criterion for closed-loop operation, administer the medicament in a limited mode; and
responsive to determining the number of BGL data points satisfies neither the criterion for closed-loop operation nor the criterion for limited operation, administer the medicament in an open-loop mode.

2. The ambulatory infusion pump of claim 1, wherein:
the processor is further configured to receive a basal rate; and
administering the medicament in the limited mode comprises (i) determining a flow rate for delivering the medicament to the user based on the BGL data points, (ii) determining whether the flow rate exceeds the basal rate, (iii) responsive to determining the flow rate does not exceed the basal rate, causing the pump mechanism to deliver the medicament to the user at the flow rate, and (iv) responsive to determining the flow rate exceeds the basal rate, causing the pump mechanism to deliver the medicament to the user at the basal rate.

3. The ambulatory infusion pump of claim 2, wherein:
administering the medicament in the closed-loop mode comprises (i) determining the flow rate based on the BGL data points and (ii) causing the pump mechanism to deliver the medicament to the user at the flow rate; and
administering the medicament in the open-loop mode comprises (i) forgoing determining the flow rate based on the BGL data points and (ii) causing the pump mechanism to deliver the medicament to the user at the basal rate.

4. The ambulatory infusion pump of claim 1, wherein the processor is further configured to:
establish a connection with an electronic device via the wireless antenna, wherein receiving the BGL data points via the wireless antenna comprises receiving the BGL data points from the electronic device;
detect a failure in the connection with the electronic device; and
responsive to detecting the failure in the connection, administer the medicament in the open-loop mode until the connection with the electronic device is reestablished.

5. The ambulatory infusion pump of claim 1, wherein the processor is further configured to:
determine an amount of noise in the BGL data points; and
adjust the amount of time, the criterion for closed-loop operation, or the criterion for limited operation based on the amount of BGL noise.

6. The ambulatory infusion pump of claim 5, wherein:
the BGL data points are collected by a CGM;
the processor is further configured to (i) receive an identifier of the CGM and (ii) determine a noise rating for the CGM based on the identifier of the CGM; and
determining the amount of noise in the BGL data points is based on the noise rating.

7. The ambulatory infusion pump of claim 5, wherein:
the BGL data points are collected by a CGM;
the processor is further configured to (i) receive an identifier of the CGM and (ii) determine a sampling rate for the CGM based on the identifier of the CGM; and
adjusting the amount of time, the criterion for closed-loop operation, or the criterion for limited operation is further based on the sampling rate.

8. The ambulatory infusion pump of claim 1, wherein:
the previous amount of time is a previous thirty minutes;
the number of the BGL data points received within the previous amount of time satisfies the criterion for closed-loop operation if the processor received three or more BGL data points in the previous thirty minutes; and
the number of the BGL data points received within the previous amount of time satisfies the criterion for limited operation if the processor received one or two BGL data points in the previous thirty minutes.

9. The ambulatory infusion pump of claim 1, wherein the number of BGL data points satisfying the criterion for limited operation but not the criterion for closed-loop operation is due to a CGM transitioning from a warmup phase into a default phase, and the BGL data points are collected by the CGM.

10. The ambulatory infusion pump of claim 1, wherein the number of BGL data points satisfying the criterion for limited operation but not the criterion for closed-loop operation is due to a failure in communication involving a CGM, and the BGL data points are collected by the CGM.

11. A non-transitory, computer-readable medium storing instructions that, when executed by a processor of an electronic device, cause the electronic device to perform operations comprising:
receiving, via a wireless antenna, BGL data points regarding a BGL of a user;
continuously determining whether a number of the BGL data points received within a previous amount of time satisfies a criterion for closed-loop operation or a criterion for limited operation;
responsive to determining the number of BGL data points satisfies the criterion for closed-loop operation, administering a medicament in a closed-loop mode;
responsive to determining the number of BGL data points satisfies the criterion for limited operation but not the criterion for closed-loop operation, administering the medicament in a limited mode; and
responsive to determining the number of BGL data points satisfies neither the criterion for closed-loop operation nor the criterion for limited operation, administering the medicament in an open-loop mode.

12. The non-transitory, computer-readable medium of claim 11, wherein:
the operations further comprise receiving a basal rate; and
administering the medicament in the limited mode comprises (i) determining a flow rate for delivering the medicament to the user based on the BGL data points, (ii) determining whether the flow rate exceeds the basal rate, (iii) responsive to determining the flow rate does not exceed the basal rate, causing a pump mechanism to deliver the medicament to the user at the flow rate, and (iv) responsive to determining the flow rate exceeds the basal rate, causing the pump mechanism to deliver the medicament to the user at the basal rate.

13. The non-transitory, computer-readable medium of claim 12, wherein:
administering the medicament in the closed-loop mode comprises (i) determining the flow rate based on the BGL data points and (ii) causing the pump mechanism to deliver the medicament to the user at the flow rate; and
administering the medicament in the open-loop mode comprises (i) forgoing determining the flow rate based on the BGL data points and (ii) causing the pump mechanism to deliver the medicament to the user at the basal rate.

14. The non-transitory, computer-readable medium of claim 11, wherein the operations further comprise:
establishing a connection with an electronic device via the wireless antenna, wherein receiving the BGL data points via the wireless antenna comprises receiving the BGL data points from the electronic device;
detecting a failure in the connection with the electronic device; and
responsive to detecting the failure in the connection, administering the medicament in the open-loop mode until the connection with the electronic device is reestablished.

15. The non-transitory, computer-readable medium of claim 11, wherein the operations further comprise:
determining an amount of noise in the BGL data points; and
adjusting the amount of time, the criterion for closed-loop operation, or the criterion for limited operation based on the amount of BGL noise.

16. The non-transitory, computer-readable medium of claim 15, wherein:
the BGL data points are collected by a CGM;
the operations further comprise (i) receiving an identifier of the CGM and (ii) determining a noise rating for the CGM based on the identifier of the CGM; and
determining the amount of noise in the BGL data points is based on the noise rating.

17. The non-transitory, computer-readable medium of claim 15, wherein:
the BGL data points are collected by a CGM;
the operations further comprise (i) receiving an identifier of the CGM and (ii) determining a sampling rate for the CGM based on the identifier of the CGM; and
adjusting the amount of time, the criterion for closed-loop operation, or the criterion for limited operation is further based on the sampling rate.

18. The non-transitory, computer-readable medium of claim 11, wherein:
the previous amount of time is a previous thirty minutes;
the number of the BGL data points received within the previous amount of time satisfies the criterion for closed-loop operation if three or more BGL data points were received in the previous thirty minutes; and
the number of the BGL data points received within the previous amount of time satisfies the criterion for limited operation if one or two BGL data points were received in the previous thirty minutes.

19. The non-transitory, computer-readable medium of claim 11, wherein the number of BGL data points satisfying the criterion for limited operation but not the criterion for closed-loop operation is due to a CGM transitioning from a warmup phase into a default phase, and the BGL data points are collected by the CGM.

20. A computer-implemented method for operating an ambulatory infusion pump in a limited mode, the method comprising:
receiving, via a wireless antenna, BGL data points regarding a BGL of a user;
continuously determining whether a number of the BGL data points received within a previous amount of time satisfies a criterion for closed-loop operation or a criterion for limited operation;
responsive to determining the number of BGL data points satisfies the criterion for closed-loop operation, administering a medicament in a closed-loop mode;
responsive to determining the number of BGL data points satisfies the criterion for limited operation but not the criterion for closed-loop operation, administering the medicament in a limited mode; and
responsive to determining the number of BGL data points satisfies neither the criterion for closed-loop operation nor the criterion for limited operation, administering the medicament in an open-loop mode.
